(19) United States
(12) Patent Application Publication
(10) Pub. No.: US 2026/0098852 A1
(43) Pub. Date: Apr. 9, 2026

(54) DRAPE TESTING APPARATUS WITH LIFTER OPERABLE TO RAISE AND DISENGAGE SUPPORT PLATE

(71) Applicant: CLO Virtual Fashion Inc., Seoul (KR)

(72) Inventors: Eun Jung JU, Seoul (KR); Kwang Yun KIM, Seoul (KR); Sung Jin YOON, Seoul (KR)

(21) Appl. No.: 19/415,647

(22) Filed: Dec. 10, 2025

Related U.S. Application Data
(63) Continuation of application No. PCT/KR2024/008294, filed on Jun. 17, 2024.

(30) Foreign Application Priority Data
Jun. 14, 2024 (KR) 10-2024-0077666
Jun. 16, 2023 (KR) 10-2023-0077228

(51) Int. Cl. G01N 33/36; G01N 21/01; G01N 21/59

(52) U.S. Cl. CPC G01N 33/367; G01N 21/01; G01N 21/59; G01N 2201/06113

(57) ABSTRACT

This drape testing apparatus comprises: a fixing body; a plate configured to support a center region of a fabric, to move in a direction opposite to a direction of gravity, and to be fixed to the fixing body; and a lifter configured to support a peripheral region different from the center region of the fabric, to move in the direction opposite to the direction of gravity, and to move in the direction of gravity while the plate is fixed to the fixing body.

### CROSS-REFERENCE TO RELATED APPLICATIONS

This is a bypass continuation-in-part of International PCT Application No. PCT/KR2024/008294, filed on June 17, 2024, which claims priority to Republic of Korea Patent Application Number 10-2023-0077228, filed on June 16, 2023 and Republic of Korea Patent Application Number June 14, 2024, which are incorporated by reference herein in their entirety.

### TECHNICAL FIELD

The disclosure relates to a drape testing apparatus for evaluating and measuring drape properties of fabrics.

### BACKGROUND ART

Fabric drape refers to the degree and manner in which a fabric deforms or changes its shape when hanging under its weight in a specific condition. The fabric drape depends on various properties of a fabric such as luster, a color, and texture, and may define the appearance of a garment formed of the fabric. U.S. Patent No. 5,097,713 discloses an apparatus for testing the stiffness of fabrics, which affects the fabric drape.

### SUMMARY

Embodiments relate to an apparatus for testing draping of a fabric. The drape testing apparatus includes one or more pillars, a lifter, and a support plate. The one or more pillars extend vertically. The lifter is slidable along the one or more pillars and includes a top surface that supports a first region of a fabric during raising of the lifter. The support plate is movable vertically and engages the lifter to rise with the lifter during raising of the lifter, but disengages from the lifter during dropping of the lifter. The support plate secures a second region of the fabric after the support plate is raised.

In one or more embodiments, the lifter includes a hole for at least partially receiving the support plate.

In one or more embodiments, the drape testing apparatus further includes a base that mounts the one or more pillars.

In one or more embodiments, the drape testing apparatus further includes a bridge suspended by ends of the one or more pillars. The support plate is secured to the bridge after the support plate is raised.

In one or more embodiments, the drape testing apparatus further includes a fixing plate under the bridge. The fixing plate has a surface facing the support plate to secure the second region of the fabric with an upper surface of the support plate.

In one or more embodiments, the fixing plate includes a magnet to secure the support plate.

In one or more embodiments, the drape testing apparatus further include one or more actuators operated to vertically raise the lifter.

In one or more embodiments, the drape testing apparatus further includes a clutch to selectively fix the lifter to the one or more pillars after the lifter is raised.

In one or more embodiments, the clutch includes an electromagnet that is activated to fix the lifter to the one or more pillars in a raised position and deactivated to release the lifter to drop from the raised position.

In one or more embodiments, the drape testing apparatus further includes a laser emitting a laser beam onto a center of the support plate.

In one or more embodiments, the drape testing apparatus further includes a camera below the support plate. The camera captures an image of the fabric.

In one or more embodiments, the drape testing apparatus further includes a texture scanner above the support plate and scans a texture of the fabric.

In one or more embodiments, the drape testing apparatus further includes a light source below the support plate and at least partially illuminates the fabric.

In one or more embodiments, the drape testing apparatus further includes a removable case that enclose the one or more pillars, the lifter and the support plate to block light.

Embodiments also relate to testing draping of a fabric. The support plate is raised by vertically raising a lifter engaging the support plate from a lowered position to a raised position. A first region of the fabric is placed on the lifter and a second region of the fabric is placed on the support plate. The second region of the fabric is secured after the support plate is raised. The support plate disengages from the lifter at the raised position. The lifter is dropped from the raised position, causing the first region of the fabric to flow downwards. An image of the fabric is captured by a camera below the lifter after the lifter is dropped.

In one or more embodiments, at least part of the support plate is received in a hole formed in the filter before raising the support plate.

In one or more embodiments, the second region of the fabric is secured by clamping the second region by a top surface of the support plate and a fixing plate under a bridge suspended by one or more pillars. The lifter is raised or dropped by sliding along the one or more pillars.

In one or more embodiments, one or more actuators are operated to raise the lifter.

In one or more embodiments, the lifter is fixed to the one or more pillars after raising the lifter by activating a clutch. The clutch is then deactivated to cause the lifter to drop.

In one or more embodiments, a laser beam is emitted onto the support plate to assist placing of the fabric onto the support plate and the lifter.

### BRIEF DESCRIPTION OF DRAWINGS

The foregoing and other aspects, features, and advantages of embodiments in the disclosure will be apparent from the following detailed description with reference to the accompanying drawings.

FIG. 1 is a perspective view of a drape testing apparatus, according to an embodiment.

FIG. 2 is a plan view of the drape testing apparatus, according to an embodiment.

FIG. 3 is a perspective view of a lifter of the drape testing apparatus, according to an embodiment.

FIG. 4 is a side view of the drape testing apparatus, according to an embodiment.

FIG. 5 is a side view of the drape testing apparatus before the lifter is raised, according to an embodiment.

FIG. 6 is a side view of the drape testing apparatus after the lifter is raised, according to an embodiment.

FIG. 7 is a perspective view of the drape testing apparatus after the lifter drops, according to an embodiment.

FIG. 8 is a perspective view of the drape testing apparatus, according to an embodiment.

FIG. 9 is an enlarged view of portion A of FIG. 4.

FIG. 10 is a schematic block diagram of an electronic device according to an embodiment.

### DETAILED DESCRIPTION

Hereinafter, embodiments will be described in detail with reference to the accompanying drawings. However, various alterations and modifications may be made to the embodiments. Here, the embodiments are not construed as limited to the disclosure. The embodiments should be understood to include all changes, equivalents, and replacements within the idea and the technical scope of the disclosure.

The terminology used herein is for the purpose of describing particular embodiments only and is not to be limiting of the embodiments. As used herein, the singular forms "a," "an," and "the" are intended to include the plural forms as well, unless the context clearly indicates otherwise. It should be further understood that the terms "comprises/comprising" and/or "includes/including" when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which embodiments belong. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

When describing the embodiments with reference to the accompanying drawings, like reference numerals refer to like constituent elements and a repeated description related thereto will be omitted. In the description of embodiments, detailed description of well-known related structures or functions will be omitted when it is deemed that such description will cause ambiguous interpretation of the present disclosure.

Also, in the description of the components, terms such as first, second, A, B, (a), (b) or the like may be used herein when describing components of the present disclosure. These terms are used only for the purpose of discriminating one constituent element from another constituent element, and the nature, the sequences, or the orders of the constituent elements are not limited by the terms. It should be noted that if one component is described as being "connected," "coupled" or "joined" to another component, the former may be directly "connected," "coupled," and "joined" to the latter or "connected", "coupled", and "joined" to the latter via another component.

The same name may be used to describe an element included in the embodiments described above and an element having a common function. Unless otherwise mentioned, the description of one embodiment may be applicable to other embodiments. Thus, duplicated description is omitted for conciseness.

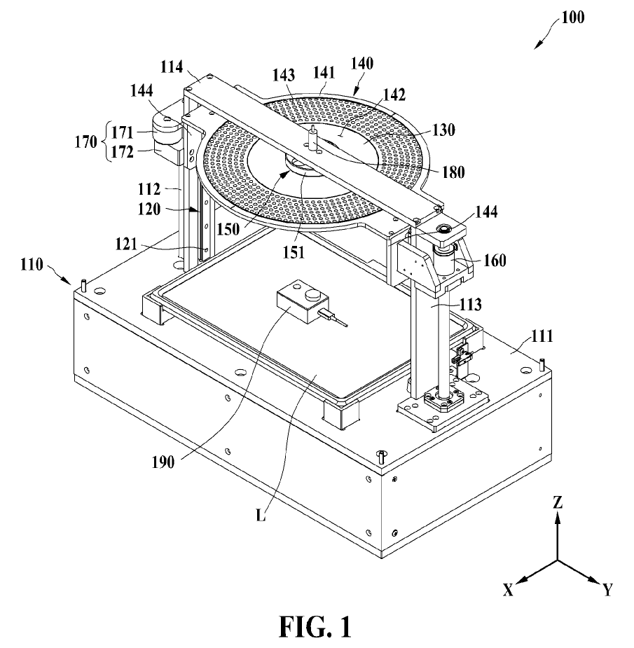

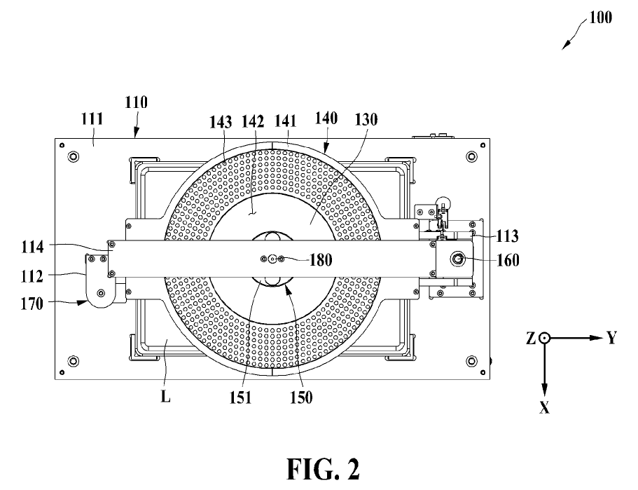

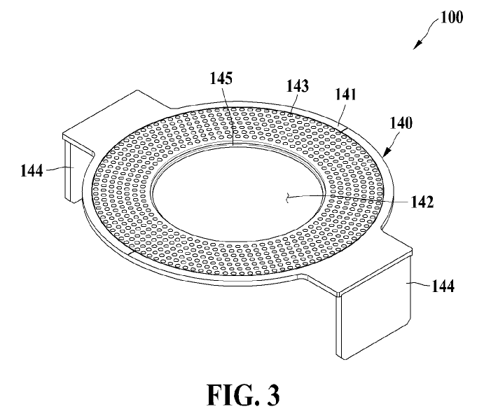

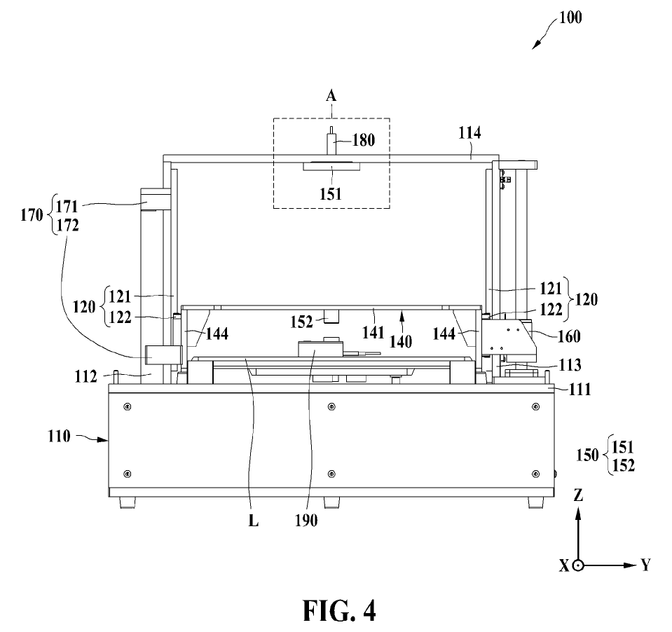

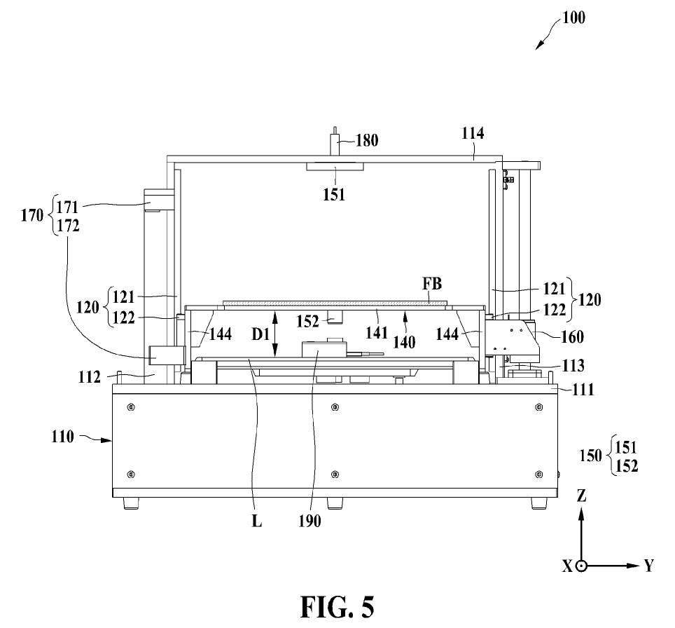

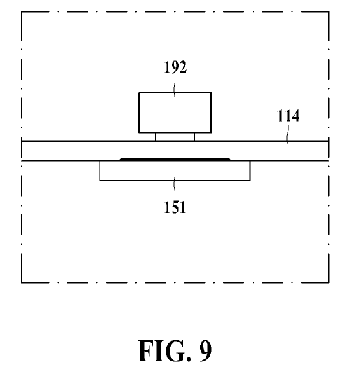

FIG. 1 is a perspective view of a drape testing apparatus 100 (hereinafter, also referred to as the "apparatus 100"), according to an embodiment. FIG. 2 is a plan view of the drape testing apparatus 100, according to an embodiment. FIG. 3 is a perspective view of a lifter 140 of the drape testing apparatus 100, according to an embodiment. FIG. 4 is a side view of the drape testing apparatus 100, according to an embodiment. FIG. 9 is an enlarged view of portion A of FIG. 4. The drape testing apparatus 100 may be used for scanning an appearance of a fabric (e.g., fabric FB of FIGS. 5 to 7) to estimate the properties of the fabric.

The properties of the fabric to be estimated include at least one of stretch-weft stiffness, stretch-wrap stiffness, shear stiffness, banding-weft stiffness, banding-wrap stiffness, and bending bias stiffness or a combination thereof. The weft indicates a horizontal thread of the fabric and is also referred to as a filling yarn. Additionally, the warp indicates a vertical thread of the fabric and is also referred to as a warping yarn.

The apparatus 100 may measure and assess an appearance change (e.g., deformation) in fabric FB due to its weight under a specific condition. The apparatus 100 may be disposed on a substantially flat surface (e.g., the ground) in an environment with the gravity.

The apparatus 100 may include, among other components, a fixing body 110, a support plate 130, and a lifter 140. The fixing body 110 is a structure that remains stationary while other components (e.g., a support plate 130 and the lifter 140) make movements relative to the fixing body 110. The fixing body 110 may include, among other components, a base 111 that supports other components (e.g., a first pillar 112, a second pillar 113, and a light source L), a first pillar 112, a second pillar 113, and a bridge 114. The base 111 may include an opening to which a fixing means (e.g., a screw) for fixing other components is secured. The base 111 may provide a support surface (e.g., a surface in the +Z normal direction) onto which other components are mounted. The base 111 may be of a plate shape, but is not limited thereto, and may be of various shapes. The base 111 may be made of metal such as aluminum.

The first pillar 112 may be mounted on a first portion (e.g., an edge portion in the -Y direction) of the base 111. The first pillar 112 may extend from the base 111 in a direction (e.g., the +Z direction) perpendicular to the base 111 and be supported by the base 111. The first pillar 112 may also be made of metal such as aluminum.

The second pillar 113 may also be mounted on the base 111 at a position opposite (e.g., an edge portion in the +Y direction) to the first pillar 112. The second pillar 113 may extend from the base 111 in a direction perpendicular to the top surface of the base 111 (e.g., the +Z direction). The second pillar 113 may be supported by the base 111. The second pillar 113 may also be made of metal such as aluminum.

In other embodiments, the fixing body 110 may include only a single pillar (e.g., the first pillar 112) or more than two pillars (e.g., the first pillar 112, the second pillar 113, a third pillar, and etc.) on different portions of the base 111.

The bridge 114 extends between the first pillar 112 and the second pillar 113. Specifically, the bridge 114 connects a first end (e.g., an end in the +Z direction) of the first pillar 112 and a first end (e.g., an end in the +Z direction) of the second pillar 113, suspended over on the base 111. Other components (e.g., the support plate 130 or the lifter 140) may be placed between the base 111 and the bridge 114. The bridge 114 may be solid (e.g., a rectangular plate) and have a cross-section of a polygonal shape. The bridge 114 may be substantially flat. The bridge 114 may be made of metal such as aluminum.

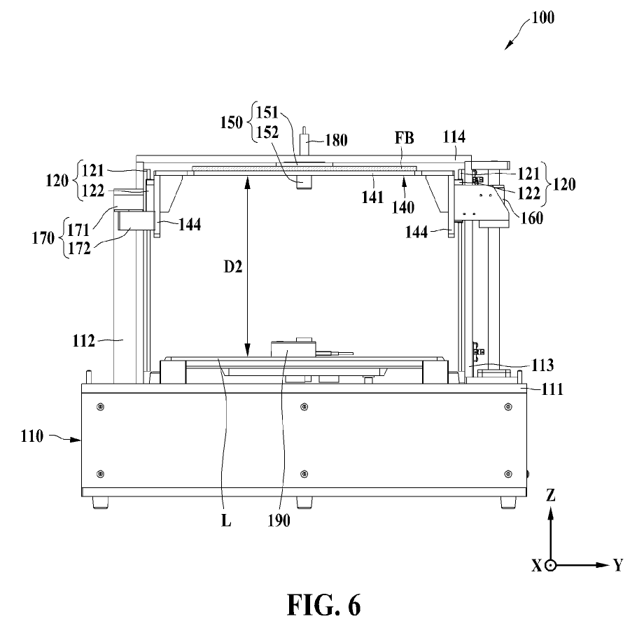

The support plate 130 provides a support surface for mounting a fabric (e.g., fabric FB of FIGS. 5 to 7). The support plate 130 may support a region (e.g., a center region) of the fabric. The support surface (e.g., a top surface) of the support plate 130 may include a solid object having a substantially circular or elliptical cross-sectional shape (e.g., a disk). In other embodiments, the support plate 130 includes a solid object having a polygonal cross-sectional shape (e.g., a rectangular plate). In yet other embodiments, the support plate 130 may be replaced with a substantially spherical or hemispherical solid object. The support surface of the support plate 130 may be substantially flat. The support plate 130 may be made of metal such as aluminum. The support plate 130 may move relative to the fixing body 110. The support plate 130 is at least partially supported by the lifter 140. The support plate 130 may move (e.g., elevate) in a direction (e.g., the +Z direction) substantially opposite to the direction of the gravity. When the support plate 130 moves in the direction (e.g., the +Z direction) substantially opposite to the direction of the gravity, the support plate 130 may be fixed to the fixing body 110. The support plate 130 may be fixed to the bridge 114 when the support plate 130 is moved to a predetermined position (e.g., second height D2 of FIG. 6). The support plate 130 may include a magnet. When the support plate 130 is raised to a predetermined height (e.g., the second height D2 of FIG. 6) from the base 111, the support plate 130 is fixed to the bridge 114 by the magnet.

The lifter 140 may move relative to the pillars 112, 113. The lifter 140 may move (e.g., elevate) in a direction (e.g., the +Z direction) substantially opposite to the direction of the gravity. or move (e.g., fall) substantially in the direction of gravity (e.g., the -Z direction). When the support plate 130 is fixed to the fixing body 110 or the bridge 114, the lifter 140 may move substantially in the direction of the gravity. The movement of the lifter 140 relative to the pillars 112, 113 may induce natural and consistent appearance changes in the fabric FB.

The lifter 140 may include a lifter plate 141. The lifter plate 141 may support a region (e.g., an adjacent region) of the fabric (e.g., the fabric FB of FIGS. 5 to 7) that is different from the region of the fabric supported by the support plate 130. The lifter plate 141 may move in the direction (e.g., the +Z direction) substantially opposite to the direction of the gravity. When the lifter plate 141 moves in the direction (e.g., the +Z direction) substantially opposite to the direction of the gravity, the support plate 130 may simultaneously move in the direction substantially opposite to the direction of the gravity. While the lifter plate 141 moves in the direction substantially opposite to the direction of the gravity, the height of a surface (e.g., a surface in the +Z normal direction) of the support plate 130 may be substantially the same as a height of a surface (e.g., a surface in the +Z normal direction) of the lifter plate 141.

The lifter plate 141 may move (e.g., fall) substantially in the direction of the gravity (e.g., the -Z direction). When the lifter plate 141 moves substantially in the direction of the gravity, the support plate 130 may be remain fixed to the bridge 114. When the lifter plate 141 moves (e.g., falls) substantially in the direction of the gravity (e.g., the -Z direction), a support force of the lifter plate 141 on the fabric may be reduced or removed, and the region (e.g., the adjacent region) of the fabric previously supported by the lifter plate 141 may hang downward in the direction of the gravity. In one or more embodiments, the lifter plate 141 may extend around a center axis (e.g., the Z axis) of the lifter plate 141. For example, the lifter plate 141 is of an annular shape having an inner edge and an outer edge. The center axis of the lifter plate 141 may substantially coincide with the center axis of the support plate 130. The lifter plate 141 may be placed outside of the support plate 130. Before the lifter plate 141 raised in the direction (e.g., the +Z direction) substantially opposite to the direction of the gravity, the position of the lifter plate 141 relative to the base 111 may be substantially the same as of the position of the support plate 130 relative to the base 111.

The lifter 140 may include a first hole 142 that accommodates at least a portion of the support plate 130. The first hole 142 may be of a substantially circular or elliptical shape. The shape of the first hole 142 is not limited to the circular or elliptical shape and may be a shape complementary to the shape of the support plate 130. The first hole 142 may be at the center region of the lifter plate 141. The first hole 142 may be defined by the inner edge of the lifter plate 141 and may penetrate a first side (e.g., a side in the +Z direction) of the lifter plate 141 through to a second side (e.g., a side in the -Z direction) at the opposite side of the first side. The center axis (e.g., the Z axis) of the first hole 142 may substantially coincide with the center axis (e.g., the Z axis) of the support plate 130.

The lifter 140 may include a plurality of second holes 143. The plurality of second holes 143 may also penetrate the first side (e.g., the side in the +Z direction) of the lifter plate 141 through to the second side (e.g., the side in the -Z direction) at the opposite side of the first side. The plurality of second holes 143 enables air to flow through. When the lifter plate 141 falls, air on the second side of the lifter plate 141 may flow through to the first side of the lifter plate 141 via the plurality of second holes 143, and thereby, reduce air resistance of the lifter plate 141. Accordingly, the fabric may show a desired shape change (e.g., a natural and consistent shape change) when part of the fabric flows or drapes downward in the direction of the gravity. The plurality of second holes 143 may be of a substantially circular or elliptical cross-sectional shape. The plurality of second holes 143 is not limited to the circular or elliptical shape and may have any shape suitable for passing air. For example, the plurality of second holes 143 may include a polygonal shape, such as a rectangle or an octagon.

The plurality of second holes 143 may be arranged around the center axis of the lifter plate 141 and in a circumferential direction of the lifter plate 141. The lifter 140 may include a plurality of rows (e.g., seven) of second holes 143 at various distances from the center axis of the lifter plate 141 between the inner edge and the outer edge of the lifter plate 141. The second holes 143 of one column may be arranged close (e.g., at a first distance) to the inner edge of the lifter plate 141. The second holes 143 of one column may be arranged apart from the outer edge of the lifter plate 141 by a distance (e.g., a second distance that is greater than the first distance). The size of each of the plurality of second holes 143 may be smaller than the size of the first hole 142.

The lifter 140 may include a plurality of leg plates 144. For example, one of the plurality of leg plates 144 may be on a first side edge of the lifter plate 141 and the other leg plate may be on a second side edge at an opposite side of the first side edge of the lifter plate 141. In some embodiments, the lifter 140 may include three or more leg plates 144 at different edges of the lifter plate 141, respectively. In alternative embodiments, the lifter 140 may include only a single leg plate 144. Each end region of the plurality of leg plates 144 may be connected to the side surface of the lifter plate 141. The plurality of leg plates 144 may be formed of metal such as aluminum. The plurality of leg plates 144 may face a corresponding pillar (e.g., the first pillar 112, the second pillar 113, or a third pillar (not shown), etc.). For example, a leg plate 144 may be disposed on the inner side (e.g., the +Y direction) of the first pillar 112 and face the first pillar 112, and the other second plate 144 may be disposed on the inner side (e.g., the -Y direction) of the second pillar 113 and face the second pillar 113. The plurality of leg plates 144 may have a surface that is in a perpendicular direction (e.g., the X direction and/or the Y direction) that is different from the perpendicular direction (e.g., the Z direction) of the surface of the lifter plate 141. The plurality of leg plates 144 may be parallel to the first pillar 112 and/or the second pillar 113.

The lifter 140 may include a protrusion 145, as shown in FIG. 3. The protrusion 145 may support the support plate 130. Before conducting a drape test, a user may place the support plate 130 on the protrusion 145. The protrusion 145 may be disposed at the center region of the lifter plate 141. The protrusion 145 may protrude from the inner edge of the lifter plate 141 toward the center axis (e.g., the Z axis) of the lifter plate 141. The protrusion 145 may be formed as a step on the inner edge of the lifter plate 141. The protrusion 145 may be formed along the edge of a lower region (e.g., an end region in the -Z axis direction) of the inner edge of the lifter plate 141. The protrusion 145 may be in the form of a step, but is not limited thereto. The protrusion 145 may be an arbitrary shape protruding toward the inner edge of the lifter plate 141.

The combined thickness (e.g., in the Z axis direction) of the protrusion 145 and the support plate 130 may be substantially the same as the thickness (e.g., thickness in the Z axis direction) of the lifter plate 141. When the protrusion 145 supports the lifter plate 141, the height of a first surface (e.g., the surface in the +Z normal direction) of the support plate 130 may be substantially the same as a height of a first surface (e.g., the surface in the +Z normal direction) of the lifter plate 141. The protrusion 145 may include a metal material. For example, the protrusion 145 may include aluminum.

The protrusion 145 may be disposed at any position in the lifter plate 141 that may support the support plate 130. For example, in an embodiment that is not shown, the protrusion 145 may be at a lower side (e.g., a side in the -Z normal direction) of the lifter plate 141 and may protrude towards the center axis of the lifter plate 141. The protrusion 145 may be configured to support the support plate 130 on the lower side of the lifter plate 141. When the protrusion 145 is disposed on the lower side of the lifter plate 141, the thickness (e.g., the thickness in the Z axis direction) of the support plate 130 may be substantially the same as the thickness (e.g., the thickness in the Z axis direction) of the lifter plate 141.

The fixing body 110, the support plate 130, and the lifter 140 may have a color having a lower saturation (e.g., black) than the color of the fabric (e.g., the fabric FB of FIGS. 5 to 7). This may increase the discernibility of the fabric for the fixing body 110, the support plate 130, and the lifter 140 by an image capturing device (e.g., the camera 190). For example, the fixing body 110, the support plate 130, and the lifter 140 may be anodized.

The apparatus 100 may include a fixing unit 150. The fixing unit 150 may fix the support plate 130 and/or the fabric (e.g., the fabric FB of FIGS. 5 to 7) to the bridge 114. For this purpose, the fixing unit 150 may include various structures (e.g., an electromagnet, an anchor, etc.) to fix the support plate 130 and/or the fabric to the bridge 114. For example, the fixing unit 150 may cause the support plate 130 to be fixed directly and magnetically to the bridge 114. For example, the fixing unit 150 may cause the support plate 130 to be fixed to the bridge 114 by magnetic coupling between other components (e.g., a fixing plate 151 and a second fixing counterpart 152). The fixing unit 150 may reduce or prevent undesired shape changes of the fabric due to the air flow around the fabric while the lifter plate 141 falls. The fixing unit 150 may fix the support plate 130 to the fixing body 110 or the bridge 114 when the support plate 130 moves to a predetermined height (e.g., a height that is substantially the same as the second height D2 of FIG. 6) from the base 111. When the support plate 130 is fixed to the fixing body 110 or the bridge 114, the lifter 140 may move substantially in the direction of the gravity (e.g., the -Z axis direction) relative to the support plate 130 with the center region of the fabric fixed to the support plate 130 while the remaining regions of the fabric flows or drapes downward.

The fixing unit 150 may include a fixing plate 151 disposed on the bridge 114. The fixing plate 151 may include a magnet. The fixing plate 151 may be disposed on a bottom surface (e.g., a surface in the -Z direction) of the bridge 114. The fixing plate 151 may be substantially disposed on the center axis (e.g., the Z axis) of the lifter plate 141. Although FIGS. 4 to 7 illustrate that the fixing plate 151 is on the bottom surface of the bridge 114, the example is not limited thereto, and the fixing plate 151 may be disposed at any position that may fix the support plate 130 and/or the fabric to the bridge 114. The fixing unit 150 may include a fixing counterpart 152 disposed on the support plate 130. The fixing counterpart 152 may include a magnet. The fixing counterpart 152 may be disposed on a bottom surface (e.g., a surface in the -Z direction) of the support plate 130. When the support plate 130 rises to a predetermined height (e.g., the height that is substantially the same as the second height D2 of FIG. 6) from the base 111, the fixing plate 151 and the fixing counterpart 152 may be magnetically coupled to each other. The support plate 130 and/or the fabric FB may be fixed between the fixing plate 151 and the fixing counterpart 152.

The plurality of linear guides 120 may guide the linear vertical movement of the lifter 140. For example, the plurality of linear guides 120 may guide the fall of the lifter 140 in the direction of the gravity. The plurality of linear guides 120 may also guide the movement of the lifter 140 in a direction opposite to the direction of the gravity.

The plurality of linear guides 120 may each include a rail 121 and a slider 122 configured to slide relative to the rail 121. One rail 121 may be disposed on the first pillar 112 and the other rail 121 may be disposed on the second pillar 113. The slider 122 may be disposed on one corresponding second plate 144.

The rail 121 may extend in a vertical direction (e.g., the Z direction) of the first pillar 112 or the second pillar 113 along the inner surface of the first pillar 112 or the second pillar 113. The slider 122 is attached to a lower region of the outer surface of the second plate 144 and enables the lifter 140 to move smoothly in vertical directions. In alternative embodiments, the rail 121 may be disposed on the second plate 144 and the slider 122 may attached to the first pillar 112 or the second pillar 113.

The apparatus 100 may include an actuator 160 that operates to raise the lifter 140 in a direction (e.g., the +Z axis direction) that is substantially opposite to the direction of the gravity. The actuator 160 may be installed on the first pillar 112 or the second pillar 113. The actuator 160 may slide the slider 122 relative to the rail 121 in a direction that is substantially opposite to the direction of the gravity. The actuator 160 may also allow the lifter 140 to free-fall substantially in the direction of the gravity (e.g., the -Z axis direction) after the lifter plate 141 is moved to a predetermined height (e.g., the second height D2 of FIG. 6). That is, the actuator 160 does not interference with the free fall of the lifter 140. For example, while the actuator 160 elevates the lifter 140, the actuator 160 may be coupled to the second plate 144 or the slider 122 but after the lifter plate 141 is moved to a predetermined height (e.g., the second height D2 of FIG. 6), the coupling may be released.

The apparatus 100 may include a clutch 170 that couples the lifter 140 to the first pillar 112 and controls the falling of the lifter 140. In a coupled state of the clutch 170, the lifter 140 may be fixed relative to the first pillar 112, and therefore to the fixing body 110, whereas in a decoupled state, the lifter 140 may move vertically relative to the first pillar 112. The clutch 170 may couple the lifter 140 to the fixing body 110 after the lifter plate 141 is moved to a predetermined height (e.g., the second height D2 of FIG. 6). When the clutch 170 decouples the lifter 140 from the first pillar 112, the lifter 140 drops. For this purpose, the clutch 170 may include a first electromagnetic element 171 and a second electromagnetic element 172 that electromagnetically couples each other. For example, the first electromagnetic element 171 and the second electromagnetic element 172 may each include an electromagnet. While current flows through each of the first electromagnetic element 171 and the second electromagnetic element 172, the first electromagnetic element 171 and the second electromagnetic element 172 may remain coupled to each other. On the other hand, when the current flowing through at least one of the first electromagnetic element 171 and the second electromagnetic element 172 is reduced or blocked, the first electromagnetic element 171 and the second electromagnetic element 172 may be separated from each other. The first electromagnetic element 171 may be installed at an upper part of one pillar (e.g., the first pillar 112 or the second pillar 113) and the second electromagnetic element 172 may be disposed on a lower part of one second plate 144 corresponding to the pillar.

In an embodiment, the clutch 170 may include an electromagnetic element and a magnetic element. For example, the electromagnetic element may include an electromagnet and a magnet element may include a permanent magnet. While the current flows through the electromagnetic element, the electromagnetic element and the magnetic element may remain coupled to each other, whereas when the current flowing through the electromagnetic element is reduced or blocked, the electromagnetic element and the magnetic element may be separated from each other. For example, the electromagnetic element may be implemented as one of elements 171 and 172 illustrated in FIGS. 1 and 4 to 7, and the magnetic element may be implemented as the other of elements 171 and 172 illustrated in FIGS. 1 and 4 to 7.

The apparatus 100 may include a laser 180. The laser 180 emits laser light towards the center axis (e.g., the Z axis) of the lifter plate 141 and/or the center axis (e.g., the Z axis) of the support plate 130 when the support plate 130 and/or the lifter 140 are at a lowered position (e.g., a position at a first height D1 in FIG. 5) before elevating. A user may place the fabric on the support plate 130 and the lifter 140 to align the center of the fabric (e.g., the fabric FB of FIGS. 5 to 7) with the center of the support plate 130 on which the laser light is incident. The laser 180 may be positioned on the bridge 114. The bridge 114 may include a hole (not shown) through which the laser light passes such that the laser 180 emits the laser light onto the center axis of the support plate 130. The fixing unit 150 may include a hole (not shown) through which the laser light passes such that the laser 180 may emit the laser light along the center axis of the support plate 130.

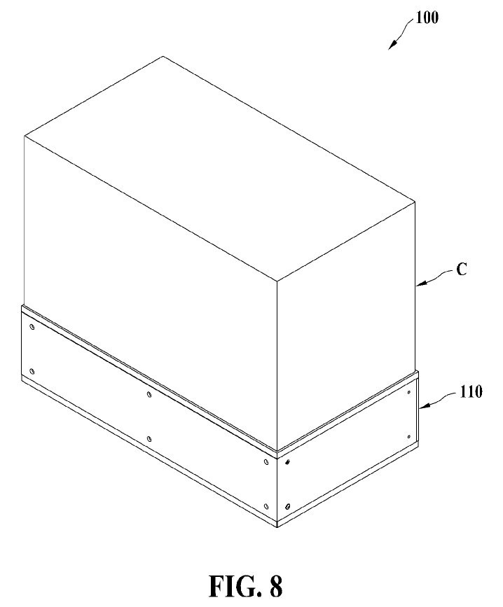

In alternative embodiments, the laser 180 may be located at any position from which the laser light is emitted towards the substantial center of the support plate 130. The bridge 114 or the fixing unit 150 may not include a hole through which the laser light passes. Although FIGS. 1-2 illustrate that the laser 180 is positioned on the center axis of the bridge 114, the laser 180 may be placed at other positions on the bridge 114. The laser 180 may be oriented to irradiate the laser light along the center axis of the support plate 130 at an arbitrary position on the bridge 114. In alternative embodiments, the laser 180 may be positioned on a component (e.g., a case C of FIG. 8) other than the bridge 114. The laser 180 positioned on the other component may be oriented to irradiate the laser light along the center axis of the support plate 130. In yet other embodiments, the apparatus 100 may not include the laser 180. Even when the apparatus 100 does not include the laser 180, the user may place the fabric (e.g., fabric FB of FIGS. 5 to 7) at a desired position on the lifter plate 141. For example, the fabric may include a mark (e.g., a "+" mark) that passes through the substantial center of the fabric, and when the user uses a portion where the mark extends and meets an edge portion of the fabric, the user may position the fabric such that the mark coincides with the substantial center of the lifter plate 141.

The apparatus 100 may include a camera 190 on the base 111. The camera 190 may be disposed between the base 111 and the support plate 130. The camera 190 may be disposed on the center axis (e.g., the Z axis) of the support plate 130. In alternative embodiments, the camera 190 may be placed at other positions to capture an image of the fabric. The camera 190 may capture the image of the fabric under the fabric (e.g., a side in the -Z direction) or the plate on the base 111. When the camera 190 is positioned below the plate, the apparatus 100 may be made more compact compared to cases where the camera 190 is positioned above the plate. The camera 190 may have a viewing angle selected to capture the image of the fabric from a relatively short distance (e.g., a distance of the second height D2 in FIG. 6). The viewing angle of the camera 190 may have an appropriate value according to a distance (e.g., the second height D2 in FIG. 6) between the elevated bridge 114 and the camera 190.

The image captured by the camera 190 may be analyzed to estimate the properties of the fabric such as the stretch-weft stiffness, the stretch-wrap stiffness, the shear stiffness, the banding-weft stiffness, the banding-wrap stiffness, and the bending bias stiffness.

Referring to FIG. 9, the apparatus 100 may further include a texture scanner 192. The texture scanner 192 may be configured to scan the texture of the fabric (e.g., fabric FB of FIGS. 5 to 7). The texture scanner 192 may scan the texture by obtaining the image of the fabric. For example, when the support plate 130 is positioned at a first height D1 from the base 111, the lifter plate 141 is also positioned at the first height D1 from the base, and the fabric FB is positioned on the plate and the lifter plate, the texture scanner 192 may scan the texture of the fabric. The texture scanner 192 may be positioned on the bridge 114. The texture scanner 192 may be positioned on the bridge 114. The texture scanner 192 may be positioned substantially on the center axis of the bridge 114. Although FIG. 9 does not illustrate the laser 180, both the laser 180 and the texture scanner 192 may be positioned on the bridge 114. The scanned texture may be used to generate a digital version of the fabric.

The apparatus 100 may include a light source L. The light source L may be on the base 111 and the camera 190 may be disposed on the light source L. The light source L may be configured to irradiate light towards the fabric (e.g., the fabric FB of FIGS. 5 to 7). The light source L may increase quality of the image capture by camera 190 to better discern the fabric. The light source L may include a light-emitting diode (LED). The light source L may enable the camera 190 to obtain an image of the fabric without shadow when the apparatus is enclosed in a case (e.g., the case C of FIG. 8) to conduct the drape test. In other embodiments, the light source L may be disposed on a region different from a region where the camera 190 is placed on the base 111. For example, the camera 190 is disposed on a center region of the base 111 while the light source L is disposed on an adjacent region of the base 111. The light source L disposed on the adjacent region may at least partially enclose the camera 190. The light source L may be configured to irradiate light towards the fabric (e.g., the fabric FB of FIGS. 5 to 7). The light source L may increase an ability of camera 190 to discern the fabric. The light source L may be embodied as a light emitting diode (LED) device.

The apparatus 100 may include at least one more light source (not shown) on the bridge 114 or the guide 120. The second light source may improve the visibility of the texture when the texture scanner 192 scans the texture of the fabric (e.g., the fabric FB of FIGS. 5 to 7). Such additional light source may include an LED device, and may enable obtaining of an image of the fabric without shadow created by the texture scanner 192 or other components (e.g., the bridge 114, etc.) while the texture scanner 192 scans the texture.

FIGS. 5 to 7 illustrate the sequence of using the drape testing apparatus 100, according to one embodiment. Referring to FIG. 5, to conduct a drape test, a user may place fabric FB on the support plate 130 and the lifter 140. The support plate 130 may be supported by the protrusion 145 (shown in FIG. 3) and may be placed inside the lifter plate 141. Initially, the support plate 130 and the lifter plate 141 may be positioned at the first height D1 from the base 111. The user may visually confirm a location on which the light emitted from the laser 180 incidents or a mark included in the fabric and may position the fabric such that the center of the fabric coincides with the center of the support plate 130. The fixing counterpart 152 may be placed on a lower part of the support plate 130. The fixing counterpart 152 may be substantially positioned on the center axis (e.g., the Z axis) of the lifter plate 141.

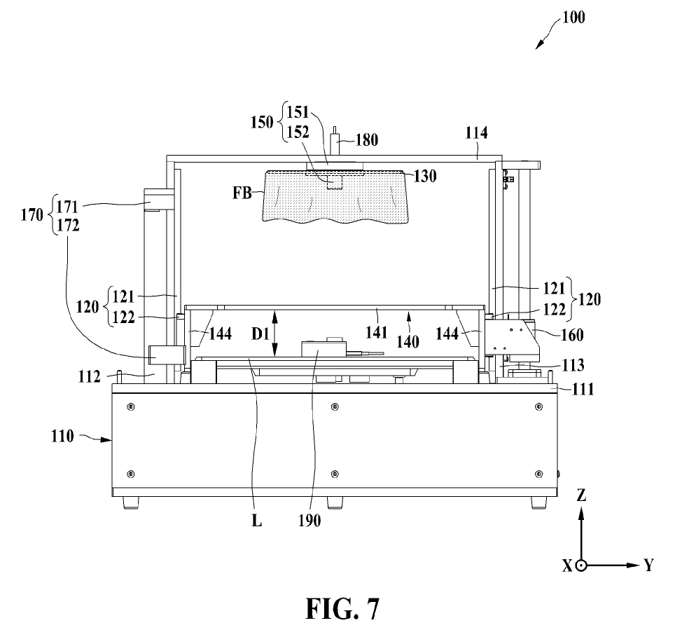

Referring to FIG. 6, the actuator 160 raises the second plate 144 in a direction opposite to the direction of gravity (e.g., the +Z direction) to position the support plate (e.g., the plate 130 of FIG. 7), the lifter plate 141, and the fabric FB at a second height D2 from the base 111. When the support plate 130 is raised to the raised position of the second height D2, the support plate 130 may be fixed to the bridge 114, for example, using the fixing plate 151 and the fixing counterpart 152 that are magnetically coupled to each other. When the support plate is substantially positioned at the second height D2, the fixing counterpart 152 may move towards the fixing plate 151 by magnetism. When the fixing counterpart 152 moves toward the fixing plate 151, the support plate 130 may also move. When the lifter plate 141 is positioned at the second height D2, the actuator 160 may no longer push the second plate 144. When the support plate and the lifter plate 141 are positioned at the second height D2, the clutch 170 may couple the fixing body 110 to the lifter 140. Even when the actuator 160 no longer pushes the second plate 144, the height of the lifter 140 may be locked into the second height D2 by the clutch 170.

Referring to FIG. 7, while the support plate 130 and the fabric FB are fixed to the bridge 114, the lifter 140 may fall substantially in the direction of the gravity (e.g., the -Z direction) relative to the support plate 130. The lifter 140 may fall until the lifter 140 is positioned at the first height D1 from the base 111. As support for a region of the fabric FB previously supported by the lifter 140 is removed, the region may flow or drape downwards, forming wrinkles in the region. The camera 190 may capture an image of the fabric FB with such wrinkles. The lifter 140 may move to the second height D2 where the support plate 130 and the fabric FB are fixed. Thereafter, the support plate 130 and the lifter 140 may slowly return to the first height D1.

Referring to FIG. 8, the apparatus 100 may include a case C configured to block light from an outside. The case C may be configured to be detachable from the fixing body 110. The user may use the case C to reduce or remove noise due to background while conducting the drape test. The case C may have a color (e.g., black) with a lower saturation than the saturation of the fabric FB. The surface of the case C may include a paint of low saturation color. This may increase the discernibility of the fabric for the case C by an optical element (e.g., the camera 190). For example, the case C may be anodized. The user may conduct the drape test without the case C. Although FIGS. 5 to 7 illustrate a process of conducting the drape test without the case C, the user may perform the drape test process shown in FIGS. 5 to 7 while coupling the case C to the fixing body 110.

As described above, although the embodiments have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

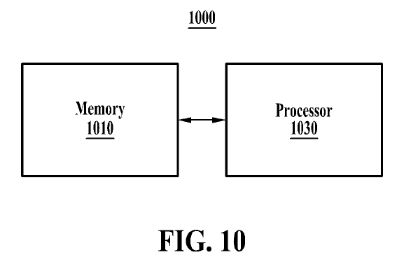

FIG. 10 is a schematic block diagram of an electronic device according to an embodiment. Referring to FIG. 10, an electronic device 1000 (e.g., the apparatus 100 of FIGS. 1 to 9) may include a memory 1010 and a processor 1030.

The memory 1010 may store instructions (or a program) executed by the processor 1030. For example, the instructions may include instructions for executing an operation of the processor 1030 and/or an operation of each component of the processor 1030.

The memory 1010 may include one or more computer readable storage media. The memory 1010 may include non-volatile storage devices (e.g., a magnetic hard disk, an optical disc, a floppy disk, flash memory, electrically programmable read-only memory (EPROM), and electrically erasable and programmable ROM (EEPROM)).

The memory 1010 may be a non-transitory media. The term "non-transitory" may indicate that the storage medium is not embodied in a carrier wave or a propagated signal. However, the term "non-transitory" should not be interpreted to mean that the memory 1010 is non-movable.

The processor 1030 may process data stored in the memory 1010. The processor may execute computer-readable code (e.g., software) stored in the memory 1010 and instructions triggered by the processor 1030.

The processor 1030 may be a hardware-implemented data-processing device including circuitry having a physical structure to perform desired operations. For example, the desired operations may include code or instructions included in the program.

For example, the hardware-implemented data-processing device may include a microprocessor, a central processing unit, a processor core, a multi-core processor, a multiprocessor, an application-specific integrated circuit (ASIC), and a field programmable gate array (FPGA).

The processor 1030 may cause the electronic device 1000 to perform one or more operations by executing the code and/or instructions stored in the memory 1010. The operations performed by the electronic device 1000 may be substantially the same as the operations performed by the apparatus 100 described with reference to FIGS. 1 to 9. Accordingly, repeated descriptions thereof are omitted.

What is claimed is:

1. A drape testing apparatus comprising:
one or more pillars extending vertically;
a lifter slidable along the one or more pillars, the lifter comprising a top surface configured to support a first region of a fabric during raising of the lifter; and
a support plate movable vertically and configured to:
engage the lifter to rise with the lifter during raising of the lifter,
disengage from the lifter during dropping of the lifter, and
secure a second region of the fabric responsive to the support plate being raised.

2. The drape testing apparatus of claim 1, wherein the lifter comprises a hole for at least partially receiving the support plate.

3. The drape testing apparatus of claim 1, further comprising a base configured to mount the one or more pillars.

4. The drape testing apparatus of claim 1, further comprising a bridge suspended by ends of the one or more pillars, wherein the support plate is secured to the bridge responsive to the support plate being raised.

5. The drape testing apparatus of claim 4, further comprising a fixing plate under the bridge, the fixing plate having a surface facing the support plate to secure the second region of the fabric with an upper surface of the support plate.

6. The drape testing apparatus of claim 5, wherein the fixing plate comprises a magnet to secure the support plate.

7. The drape testing apparatus of claim 1, further comprising one or more actuators operated to vertically raise the lifter.

8. The drape testing apparatus of claim 1, further comprising a clutch configured to selectively fix the lifter to the one or more pillars responsive to raising the lifter.

9. The drape testing apparatus of claim 8, wherein the clutch comprises an electromagnet that is activated to fix the lifter to the one or more pillars in a raised position and deactivated to release the lifter to drop from the raised position.

10. The drape testing apparatus of claim 1, further comprising a laser emitting a laser beam onto a center of the support plate.

11. The drape testing apparatus of claim 1, further comprising a camera below the support plate and configured to capture an image of the fabric.

12. The drape testing apparatus of claim 1, further comprising a texture scanner above the support plate and configured to scan a texture of the fabric.

13. The drape testing apparatus of claim 1, further comprising a light source below the support plate and configured to at least partially illuminating the fabric.

14. The drape testing apparatus of claim 1, further comprising a removable case configured to enclose the one or more pillars, the lifter and the support plate to block light.

15. A method of testing draping of a fabric, comprising:
raising a support plate by vertically raising a lifter engaging the support plate from a lowered position to a raised position, a first region of the fabric placed on the lifter and a second region of the fabric placed on the support plate;
securing the second region of the fabric responsive to the support plate being raised;
disengaging the support plate from the lifter at the raised position;
dropping the lifter from the raised position, causing the first region of the fabric to flow downwards; and
capturing an image of the fabric by a camera below the lifter, responsive to dropping the lifter.

16. The method of claim 15, further comprising receiving at least part of the support plate in a hole formed in the lifter before raising the support plate.

17. The method of claim 15, wherein the second region of the fabric is secured by clamping the second region by a top surface of the support plate and a fixing plate under a bridge suspended by one or more pillars, the lifter raised or dropped by sliding along the one or more pillars.

18. The method of claim 17, further comprising operating one or more actuators to raise the lifter.

19. The method of claim 15, further comprising:
fixing the lifter to one or more pillars along which the lifter slides responsive to raising the lifter by activating a clutch; and
deactivating the clutch to cause the lifter to drop.

20. The method of claim 15, further comprising emitting a laser beam onto the support plate to assist placing of the fabric onto the support plate and the lifter.
